(19) United States
(12) Patent Application Publication
(10) Pub. No.: US 2021/0087195 A1
(43) Pub. Date: Mar. 25, 2021

(54) NEW CRYSTAL FORM OF ACALBRUTINIB, PREPARATION METHOD THEREFOR AND USE THEREOF

(71) Applicant: Crystal Pharmaceutical (Suzhou) Co., Ltd., Suzhou (CN)

(72) Inventors: Minhua Chen, Suzhou (CN); Yanfeng Zhang, Suzhou (CN); Yuan Liu, Suzhou (CN); Long Zhang, Suzhou (CN); Chaohui Yang, Suzhou (CN)

(21) Appl. No.: 17/050,329

(22) PCT Filed: Mar. 5, 2019
(86) PCT No.: PCT/CN2019/076944

§ 371 Date: Oct. 23, 2020

(30) Foreign Application Priority Data
Apr. 26, 2018 (CN) 201810387686.2

(51) Int. Cl. C07D 487/04

(52) U.S. Cl. CPC C07D 487/04; C07B 2200/13

(57) ABSTRACT

The present disclosure relates to novel crystalline forms of acalabrutinib and processes for preparation thereof. The present disclosure also relates to pharmaceutical compositions containing acalabrutinib, use of acalabrutinib for preparing Bruton's tyrosine kinase inhibitor drugs, and use of acalabrutinib for preparing drugs treating mantle cell lymphoma. The crystalline forms of the present disclosure have one or more improved properties compared with prior art, and have significant value for future drug optimization and development.

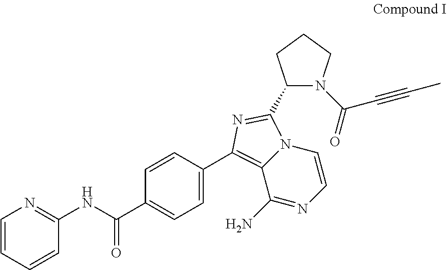 [SMILES: CC#CC(=O)N1CCC[C@H]1c1nc(-c2ccc(C(=O)Nc3ccccn3)cc2)c2c(N)nccn12]

### TECHNICAL FIELD

The present disclosure relates to the field of pharmaceutical chemistry, particularly relates to crystalline forms of acalabrutinib, processes for preparation and use thereof.

### BACKGROUND

Mantle Cell Lymphoma is a type of non-Hodgkin's lymphoma and is a hard-to-treat lymphoma. BTK is a member of the Tec family of tyrosine kinases and has been shown as a key regulator of early B cell development as well as activation and survival of mature B cells. BTK has been reported to play a role in apoptosis, and thus BTK inhibitors are useful in the treatment of certain B-cell lymphomas and leukemias.

Acalabrutinib is a second-generation BTK inhibitor with higher selectivity and lower side effects compared with the first-generation BTK inhibitor ibrutinib. The approval of acalabrutinib provides a new treatment option for patients with relapsed drug-resistant mantle cell lymphoma. Acalabrutinib was developed by Acerta and approved in the US in October 2017. The chemical name of Acalabrutinib is (S)-4-(8-amino-3-(1-(but-2-ynoyl)pyrrolidin-2-yl)imidazo[1,5-a]pyrazin-1-yl)-N-(pyridin-2-yl)benzamide (hereinafter referred to as “compound I”), and the structure is shown as follows:

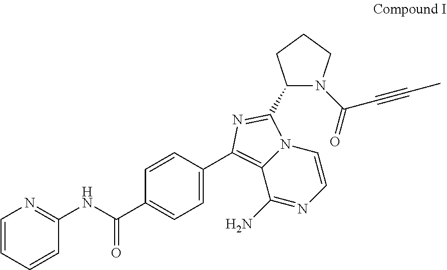 [SMILES: CC#CC(=O)N1CCC[C@H]1c1nc(-c2ccc(C(=O)Nc3ccccn3)cc2)c2c(N)nccn12]

Crystalline forms are different solids formed by different arrangement of compound molecules in the lattice space. Polymorphism is the ability of a compound to exist in two or more than two crystalline forms.

Different crystalline forms with different physicochemical properties of the same drug substance may have different in vivo dissolution and absorption, which will further affect drug's clinical efficacy and safety to some extent. In particular, for poorly soluble solid oral drugs, the above effects of the crystalline form will be greater. Therefore, drug polymorphism is an important part of drug research, and an important part of drug quality control.

WO2017002095A1 disclosed eight crystalline forms of acalabrutinib. It disclosed that crystalline form I is an anhydrate. Crystalline form II is a trihydrate with poor flowability and non-uniform particle size. Water content in crystalline form II varies with conditions and the water content can be up to 10%. Crystalline form III is an unstable dihydrate. The water content in crystalline form III varies with conditions and can be up to 8%. Crystalline form IV and crystalline form V are unstable anhydrates.

Crystalline form IV was obtained by dehydration of Crystalline form II at low relative humidity (RH) and crystalline form V was obtained by heating and dehydration of Crystalline form II. Crystalline form VI and crystalline form VII are methanol solvates; crystalline form VIII is an acetic acid solvate.

The inventors of the present disclosure surprisingly discovered crystalline form K1 of acalabrutinib, which has advantages in physiochemical properties, formulation processability and bioavailability, for example, crystalline form K1 has advantages in at least one aspect of melting point, solubility, hygroscopicity, purification ability, stability, adhesiveness, compressibility, flowability, in vitro and in vivo dissolution, and bioavailability, etc. In particular, crystalline form K1 has simple preparation process, good repeatability, good physical stability of drug substance, high solubility in organic solvents, good compressibility, good adhesiveness, good formulation stability, and high dissolution, which provides a new and better choice for the development of acalabrutinib and is of great significance.

### SUMMARY

The main objective of the present disclosure is to provide novel crystalline forms of acalabrutinib, processes for preparation and use thereof.

According to the objective of the present disclosure, crystalline form K1 of compound I is provided (hereinafter referred to as Form K1).

According to one aspect of the present disclosure, the X-ray powder diffraction pattern of Form K1 shows characteristic peaks at 2theta values of 5.8°±0.2°, 9.5°±0.2° and 14.3°±0.2° using CuKα radiation.

Furthermore, the X-ray powder diffraction pattern of Form K1 shows one or two or three characteristic peaks at 2theta values of 13.8°±0.2°, 12.8°±0.2° and 18.4°±0.2°. Preferably, the X-ray powder diffraction pattern of Form K1 shows characteristic peaks at 2theta values of 13.8°±0.2°, 12.8°±0.2° and 18.4°±0.2°.

Furthermore, the X-ray powder diffraction pattern of Form K1 shows one or two or three characteristic peaks at 2theta values of 16.3°±0.2°, 6.9°±0.2° and 11.5°±0.2°. Preferably, the X-ray powder diffraction pattern of Form K1 shows characteristic peaks at 2theta values of 16.3°±0.2°, 6.9°±0.2° and 11.5°±0.2°.

According to another aspect of the present disclosure, the X-ray powder diffraction pattern of Form K1 shows three or four or five or six or seven or eight or nine or ten or eleven or twelve or thirteen characteristic peaks at 2theta values of 5.8° 0.2°, 9.5° 0.2°, 14.3°±0.2°, 13.8°±0.2°, 12.8°±0.2°, 18.4°±0.2°, 16.3°±0.2°, 6.9°±0.2°, 11.5°±0.2°, 8.1°±0.2°, 17.7°±0.2°, 23.5°±0.2° and 24.6°±0.2° using CuKα radiation.

Without any limitation being implied, the X-ray powder diffraction pattern of Form K1 is substantially as depicted in FIG. 1.

According to the objective of the present disclosure, a process for preparing Form K1 is also provided. The process comprises:

(1) Adding acalabrutinib freebase and acid in a mixture of ketones and water, stirring, separating and drying to obtain a solid, suspending the above solid in water and adding an alkaline solution into the suspension while stirring, separating the solid to obtain Form K1; or

(2) Adding acalabrutinib free base in acid solution, stirring, separating and drying to obtain a solid, suspending the solid in alkaline solution, separating the solid to obtain Form K1.

Preferably:

In method (1), said acid is maleic acid or fumaric acid. Said alkaline solution is NaOH aqueous solution. Said ketone solvent are acetone, 2-butanone or methyl isobutyl ketone.

In method (2), said acid solution is aqueous solution of HCl. Said alkaline solution is aqueous solution of NaOH.

Form K1 of the present disclosure has the following advantages:

(1) Compared with Form I of WO2017002095A1, Form K1 of the present disclosure has good physical stability in water. The solid state of Form K1 doesn't change after stirred magnetically or shaken for 7 days at room temperature and 5° C. in water, while Form I shows crystallinity decrease and almost converts into amorphous after magnetic stirring for 4 hours.

The physical stability of crystalline form in water is very important for drug production and in vivo drug absorption. In the process of drug production, solution crystallization is the most popular method, wherein water is a commonly used solvent. Good physical stability of the drug substance in water is conductive to the quality control of the product in the production process. In addition, as the crystalline drug substance has good physical stability when contacts with water, the control requirements of water content and humidity in the production process is low, and the production cost can be reduced.

Furthermore, water is the main component of biological medium in human body. Good physical stability of the drug substance in water can avoid the crystal transformation of active ingredients in human body. Crystal transformation can lead to changes in the absorption of the drug, affect bioavailability, and even cause toxicity and side effects.

(2) Form K1 of the present disclosure is thermodynamically more stable than prior art in water at room temperature and 5° C. When the solid mixture of Form K1 and Form I of WO2017002095A1 was stirred in water at room temperature and 5° C., Form I will convert into Form K1 of the present disclosure.

Thermodynamically metastable crystalline forms tend to spontaneously transform into the thermodynamically more stable crystalline form. Drug preparation and manufacturing processes, such as tableting, milling, wet granulation and freeze-drying, will accelerate the crystal transformation of thermodynamically metastable crystalline forms. In addition, the existence of thermodynamically more stable crystalline form as seeds can induce and accelerate the transformation of thermodynamically metastable state to thermodynamically more stable state. the thermodynamically more stable crystalline form is usually selected for drug substance to avoid the effect of crystal transformation on the efficacy. Compared with Form I of the prior art, Form K1 of the present disclosure is thermodynamically more stable, can reduce the above mentioned risks, and is more suitable for drug substance.

(3) Form K1 drug substance of the present disclosure has good stability under both long-term and accelerated conditions. Form K1 drug substance keeps stable for at least one month at 25° C./60% RH. The result shows that Form K1 has good stability under long-term condition and is beneficial to drug storage. Meanwhile, Form K1 keeps stable for at least one month at 40° C./75% RH. The result shows that Form K1 has good stability under accelerated condition. Drug substance will go through stress conditions caused by weather, season and regional climate differences during storage, transportation, and production processes. Form K1 drug substance has good stability under accelerated condition, which is beneficial to drug storage under special environmental conditions, for example high temperature and high humidity conditions. Form K1 drug product has good stability under both long-term and accelerated conditions. Drug product prepared from Form K1 and excipients is stable for at least 6 months at 25° C./60% RH (sealed) and 40° C./75% RH (sealed).

Crystal transformation can lead to changes in the absorption of the drug, affect bioavailability, and even cause toxicity and side effects. Form K1 has good physical stability, ensuring consistent and controllable quality of the drug substance and drug product, minimizing quality change, bioavailability change and toxicity due to crystal transformation.

(4) Form K1 of the present disclosure has good physical stability under grinding. Grinding and pulverization are often required in the drug production process. Good physical stability of the drug substance under grinding can reduce the risk of crystallinity decrease and crystal transformation during the drug production process.

(5) Compared with prior art, Form K1 of the present disclosure has higher solubilities in multiple buffers. Compared with Form I of WO2017002095A1, Form K1 has higher solubilities in pH 7.0, 7.4 and 8.7 buffers.

Acalabrutinib belongs to BCS class II and has high permeability and low solubility. Improving solubility is very important for acalabrutinib, higher solubility is beneficial to improve drug's in vivo absorption and bioavailability, thus improving drug efficacy. In addition, drug dose reduction without affecting efficacy is possible due to higher solubility, thereby reducing the drug's side effects and improving drug safety.

(6) Compared with prior art, Form K1 of the present disclosure has higher solubilities in organic solvents. Compared with Form I of WO2017002095A1, Form K1 has higher solubilities in commonly used organic solvents in crystallization, including methanol, ethanol, isopropanol, n-propanol, n-butanol, acetone, methyl isobutyl ketone, methyl ethyl ketone, ethyl acetate, tetrahydrofuran, 2-methyltetrahydrofuran, 1,4-dioxane and acetonitrile.

Form K1 of the present disclosure has higher solubilities in multiple organic solvents, which provides more choices of solvents in the crystallization process, reduces the amount of solvents used in the crystallization process, reduces the cost, and is more environmentally friendly.

(7) Form K1 drug product has a high in vitro dissolution and dissolution rate in 0.1 mol/L HCl aqueous solution. A dissolution of 84% was achieved within 20 minutes. Drug with different crystalline forms may lead to different in vivo dissolution rates, which directly affects drug's in vivo absorption, distribution, excretion and metabolism, and finally leads to difference in clinical efficacy due to different bioavailability. Dissolution and dissolution rates are important prerequisites for drug absorption. Good in vitro dissolution leads to higher in vivo absorption, better in vivo exposure, thereby improving drug's bioavailability and efficacy. High dissolution rate is beneficial for the drug to achieve peak concentration in plasma quickly after administration, thus ensuring rapid drug action.

Furthermore, Form K1 of the present disclosure also has the following advantages:

(1) Compared with prior art, Form K1 of the present disclosure has better compressibility. Failure in hardness/friability test and tablet crack issue can be avoided due to better compressibility, making the preparation process more reliable, improving product appearance and product quality. Better compressibility can increase the compression rate, thus further increases the efficiency of process and reduces the cost of compressibility improving excipients.

(2) Compared with prior art, Form K1 of the present disclosure shows superior adhesiveness. Adhesiveness evaluation results indicate that adhesion quantity of Form K1 is lower than that of prior art forms. Due to superior adhesiveness of Form K1, adhesion to roller and tooling during dry-granulation and compression process can be reduced, which is also beneficial to improve product appearance and weight variation. In addition, superior adhesiveness of Form K1 can reduce the agglomeration of drug substance, which is beneficial to the dispersion of drug substance and reduce the adhesion between drug substance and instruments, and improves the blend uniformity and content uniformity of drug product.

According to the objective of the present disclosure, a pharmaceutical composition is provided. Said pharmaceutical composition comprises a therapeutically effective amount of Form K1 and pharmaceutically acceptable carriers, diluents or excipients.

Furthermore, the use of Form K1 of the present disclosure for preparing Bruton's tyrosine kinase inhibitor drug is provided.

Furthermore, the use of Form K1 of the present disclosure for preparing drugs treating mantle cell lymphoma and/or chronic lymphocytic leukemia and/or macroglobulinemia and/or follicular lymphoma and/or diffuse large B cell lymphoma and/or multiple myeloma is provided.

According to the objective of the present disclosure, crystalline form CS10 of compound I isopropanol solvate is provided (hereinafter referred to as Form CS10).

According to one aspect of the present disclosure, the X-ray powder diffraction pattern of Form CS10 shows characteristic peaks at 2theta values of 8.5°±0.2°, 6.0°±0.2° and 18.2°±0.2° using CuKα radiation.

Furthermore, the X-ray powder diffraction pattern of Form CS10 shows one or two or three characteristic peaks at 2theta values of 20.1°±0.2°, 14.9°±0.2° and 15.3°±0.2°.

Preferably, the X-ray powder diffraction pattern of Form CS10 shows characteristic peaks at 2theta values of 20.1°±0.2°, 14.9°±0.2° and 15.3°±0.2°.

Furthermore, the X-ray powder diffraction pattern of Form CS10 shows one or two or three characteristic peaks at 2theta values of 15.9°±0.2°, 26.0°±0.2° and 26.6°±0.2°. Preferably, the X-ray powder diffraction pattern of Form CS10 shows characteristic peaks at 2theta values of 15.9°±0.2°, 26.0°±0.2° and 26.6°±0.2°.

According to another aspect of the present disclosure, the X-ray powder diffraction pattern of Form CS10 shows three or four or five or six or seven or eight or nine characteristic peaks at 2theta values of 8.5°±0.2°, 6.0°±0.2°, 18.2°±0.2°, 20.1°±0.2°, 14.9°±0.2°, 15.3°±0.2°, 15.9°±0.2°, 26.0°±0.2° and 26.6°±0.2° using CuKα radiation. Without any limitation being implied, the X-ray powder diffraction pattern of Form CS10 is substantially as depicted in FIG. 20.

According to the objective of the present disclosure, a process for preparing Form CS10 is also provided. The process comprises: Adding acalabrutinib freebase into a mixture of acetonitrile and alcohols, stirring at 10° C.-60° C. and isolating to obtain a solid, suspending the obtained solid into isopropyl acetate or a mixture of isopropyl acetate and alkyl halides, stirring at 0° C.-40° C., and isolating the solid to obtain Form CS10.

Furthermore:

Said 10° C.-60° C. is preferably 50° C. Said 0° C.-40° C. is preferably 5° C. Said volume ratio of isopropyl acetate to alkyl halide is preferably 99:1-90:10, more preferably 95:5. Said volume ratio of acetonitrile to alcohol is preferably 99:1-80:20, more preferably 90:10.

Furthermore:

Said alcohol is preferably methanol. Said alkyl halide is preferably dichloromethane. In the present disclosure “room temperature” is not a specific temperature, but a temperature range of 10−30° C.

Said “separation” is accomplished by using a conventional method in the field such as centrifugation or filtration. The operation of “centrifugation” is as follows: the sample to be separated is placed into the centrifuge tube, and then centrifuged at a rate of 10000 r/min until the solid all sink to the bottom of the tube.

In the present disclosure, “crystal” or “crystalline form” refers to the crystal or the crystalline form being identified by the X-ray diffraction pattern shown herein. Those skilled in the art are able to understand that physicochemical properties discussed herein can be characterized. The experimental errors depend on the instrument conditions, the sample preparation and the purity of samples. In particular, those skilled in the art generally know that the X-ray diffraction pattern typically varies with the experimental conditions. It is necessary to point out that, the relative intensity of the diffraction peaks in the X-ray diffraction pattern may also vary with the experimental conditions; therefore, the order of the diffraction peak intensities cannot be regarded as the sole or decisive factor. In fact, the relative intensity of the diffraction peaks in the X-ray powder diffraction pattern is related to the preferred orientation of the crystals, and the diffraction peak intensities shown herein are illustrative and identical diffraction peak intensities are not required. In addition, the experimental error of the diffraction peak position is usually 5% or less, and the error of these positions should also be taken into account. An error of ±0.2° is usually allowed. In addition, due to experimental factors such as sample thickness, the overall offset of the diffraction peak is caused, and a certain offset is usually allowed. Thus, it will be understood by those skilled in the art that a crystalline form of the present disclosure is not necessarily to have the exact same X-ray diffraction pattern of the example shown herein. Any crystalline forms whose X-ray diffraction patterns have the same or similar characteristic peaks should be within the scope of the present disclosure. Those skilled in the art can compare the patterns shown in the present disclosure with that of an unknown crystalline form in order to identify whether these two groups of patterns reflect the same or different crystalline forms.

In some embodiments, Form K1 of the present disclosure is pure and substantially free of any other crystalline forms. In the present disclosure, the term “substantially free” when used to describe a novel crystalline form, it means that the content of other crystalline forms in the novel crystalline form is less than 20% (w/w), specifically less than 10% (w/w), more specifically less than 5% (w/w) and further more specifically less than 1% (w/w).

In the present disclosure, the term “about” when referring to a measurable value such as weight of a compound or formulation, time, temperature, and the like, is meant to encompass variations of ±10%, ±5%, ±1%, ±0.5%, or even ±0.1% of the specified amount.

### BRIEF DESCRIPTION OF THE DRAWINGS

FIG. 1 shows an XRPD pattern of Form K1 in Example 1

FIG. 2 shows a DSC curve of Form K1 in Example 1

FIG. 3 shows a TGA curve of Form K1 in Example 1

FIG. 4 shows a ¹H NMR spectrum of Form K1 in Example 2

FIG. 5 shows an XRPD pattern overlay of Form K1 from physical stability study during magnetic stirring at room temperature (from top to bottom: initial, stirred for 4 hours, 1 day, 2 days and 7 days)

FIG. 6 shows an XRPD pattern overlay of Form K1 from physical stability study during magnetic stirring at 5° C. (from top to bottom: initial, stirred for 4 hours, 1 day, 2 days and 7 days)

FIG. 7 shows an XRPD pattern overlay of Form K1 from physical stability study during shaking at room temperature (from top to bottom: initial, shook for 4 hours, 1 day, 2 days and 7 days)

FIG. 8 shows an XRPD pattern overlay of Form K1 from physical stability study during shaking at 5° C. (from top to bottom: initial, shook for 4 hours, 1 day, 2 days and 7 days)

FIG. 9 shows an XRPD pattern overlay of Form I of WO2017002095A1 from physical stability study during magnetic stirring at room temperature (from top to bottom: initial, stirred for 4 hours)

FIG. 10 shows an XRPD pattern overlay of Form I of WO2017002095A1 from physical stability study during magnetic stirring at 5° C. (from top to bottom: initial, stirred for 4 hours and 1 day)

FIG. 11 shows an XRPD pattern overlay of Form I of WO2017002095A1 from physical stability study during shaking at room temperature (from top to bottom: initial, shook for 4 hours and 1 day)

FIG. 12 shows an XRPD pattern overlay of Form I of WO2017002095A1 from physical stability study during shaking at 5° C. (from top to bottom: initial, shook for 4 hours, 1 day, 2 days and 7 days)

FIG. 13 shows an XRPD pattern overlay of Form K1 and Form I of WO2017002095A1 from slurry competition experiments at 5° C. (from top to bottom: initial mixture of Form K1 and Form I, mixture of Form K1 and Form I after stirred for 6 days, Form K1, Form I)

FIG. 14 shows an XRPD pattern overlay of Form K1 and Form I of WO2017002095A1 from slurry competition experiments at room temperature (from top to bottom: initial mixture of Form K1 and Form I, mixture of Form K1 and Form I after stirred for 24 days, Form K1, Form I)

FIG. 15 shows an XRPD pattern overlay of Form K1 from stability study (from top to bottom: initial, stored at 25° C./60% RH (sealed) for one month, stored at 25° C./60% RH (open) for one month, stored at 40° C./75% RH (sealed) for one month, stored at 40° C./75% RH (open) for one month)

FIG. 16 shows an XRPD pattern overlay of Form K1 from mechanical stability study (top: before grinding, bottom: after grinding)

FIG. 17 shows an XRPD pattern overlay of Form K1 during production process (from top to bottom: capsule drug product containing Form K1, excipients and Form K1).

FIG. 18 shows a dissolution profile of Form K1 drug product

FIG. 19 shows an XRPD pattern overlay of Form K1 drug product from stability study (from top to bottom: initial, stored under 25° C./60% RH (sealed) for 6 months with 1 g of desiccant, stored under 40° C./75% RH (sealed) for 6 months with 1 g of desiccant)

FIG. 20 shows an XRPD pattern of Form CS10 in Example 14

FIG. 21 shows an XRPD pattern of Form CS10 in Example 15

### DETAILED DESCRIPTION

The present disclosure is further illustrated by the following examples which describe the preparation and use of the crystalline forms of the present disclosure in detail. It is obvious to those skilled in the art that many changes in the materials and methods can be accomplished without departing from the scope of the present disclosure.

The abbreviations used in the present disclosure are explained as follows:

XRPD: X-ray Powder Diffraction

DSC: Differential Scanning Calorimetry

TGA: Thermo Gravimetric Analysis

¹H NMR: Proton Nuclear Magnetic Resonance

HPLC: High Performance Liquid Chromatography

Instruments and methods used for data collection:

X-ray powder diffraction patterns in the present disclosure were acquired by a Bruker

D2 PHASER X-ray powder diffractometer. The parameters of the X-ray powder diffraction method of the present disclosure are as follows:

X-ray Reflection: Cu, Kα

Kα1 (Å): 1.54060; Kα2 (A): 1.54439

Kα2/Kal intensity ratio: 0.50

Voltage: 30 (kV)

Current: 10 (mA)

Scan range: from 3.0 degree to 40.0 degree

Differential scanning calorimetry (DSC) data in the present disclosure were acquired by a TA Q2000. The parameters of the DSC method of the present disclosure are as follows:

Heating rate: 10° C./min

Purge gas: nitrogen

Thermo gravimetric analysis (TGA) data in the present disclosure were acquired by a TA Q500. The parameters of the TGA method of the present disclosure are as follows:

Heating rate: 10° C./min

Purge gas: nitrogen

Proton nuclear magnetic resonance spectrum data (¹H NMR) were collected from a Bruker Avance II DMX 400M HZ NMR spectrometer. 1-5 mg of sample was weighed and dissolved in 0.5 mL of deuterated dimethyl sulfoxide to obtain a solution with a concentration of 2-10 mg/mL.

High Performance Liquid Chromatography (HPLC) data in the present disclosure were collected from an Agilent 1260, the parameters for purity test in the present disclosure are as follows:

1. Column: Ultimate LP-C18, 250×4.6 mm, 5 μm

2. Mobile Phase: A: 0.1% Phosphoric acid in H₂O (pH=3.5, TEA)
B: Acetonitrile

Gradient:

| Time (min) | % B |
|---|---|
| 0.0 | 20 |
| 9.0 | 34 |
| 11.0 | 40 |
| 18.0 | 50 |
| 22.0 | 70 |
| 30.0 | 70 |
| 31.0 | 20 |
| 40.0 | 20 |

3. 1.0 mL/min

4. Injection Volume: 10 μL

5. Detection wavelength: 230 nm

6. Column Temperature: 40° C.

7. Diluent: Acetonitrile

According to the present disclosure, acalabrutinib and/or its salt used as a raw material is solid (crystalline or amorphous), semisolid, wax or oil. Preferably, compound I and/or its salt used as a raw material is a powder solid.

Acalabrutinib free base solid used in the following examples were prepared by known methods in the prior art, for example, the method disclosed in WO2017002095A1.

### EXAMPLES

### Example 1 Preparation of Form K1

200.1 mg of acalabrutinib freebase and 51.8 mg of maleic acid were weighed into a 5-mL glass vial. 4 mL of acetone/water (95:5, v/v) was added. After being stirred at room temperature overnight, 218.0 mg of dry solid was obtained through vacuum filtration followed by vacuum drying at room temperature.

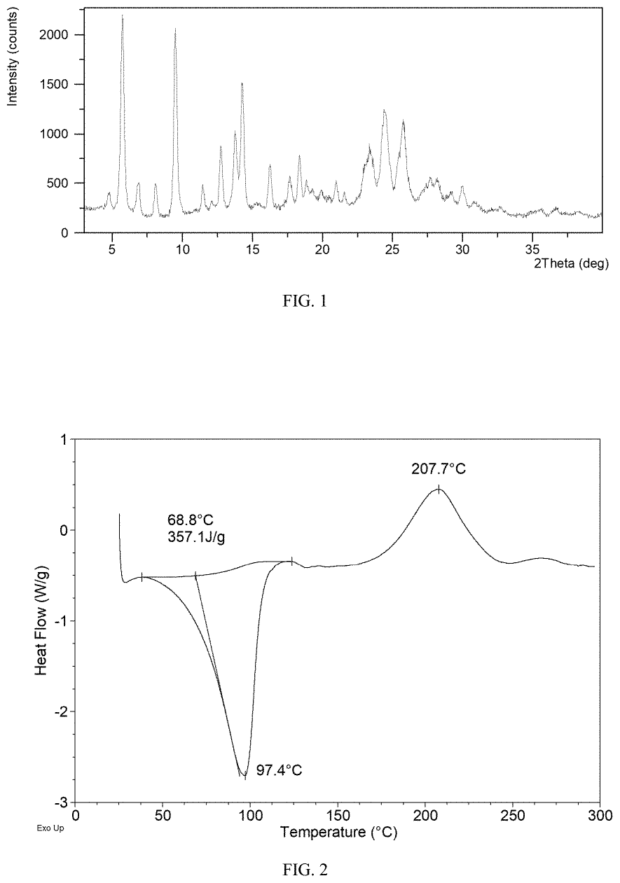

118.1 mg of the above solid was weighed into a 3-mL glass vial, and 2 mL of water was added. The mixture was stirred at room temperature to form a suspension. 22.3 mg of NaOH was dissolved in 1 mL of water to get an aqueous solution of NaOH. The aqueous solution of NaOH was added into the above suspension at room temperature. After stirring, a solid was obtained by isolation. The solid obtained in the present example was confirmed to be Form K1. The XRPD pattern of Form K1 is substantially as depicted in FIG. 1, and the XRPD data are listed in Table 1.

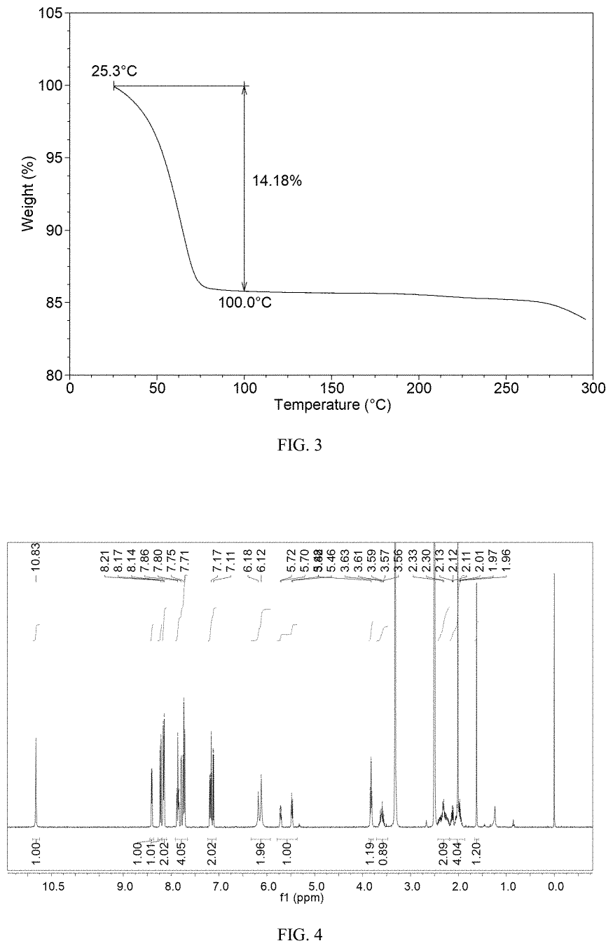

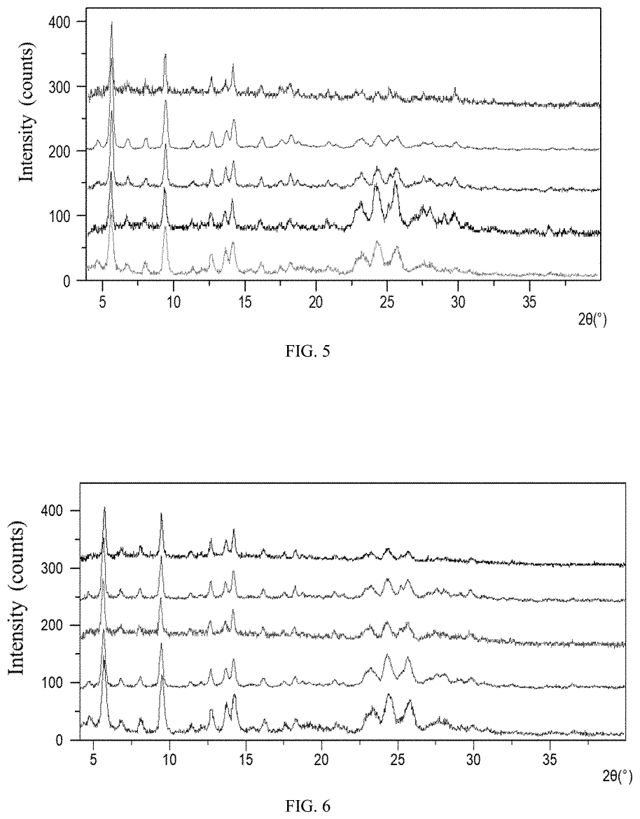

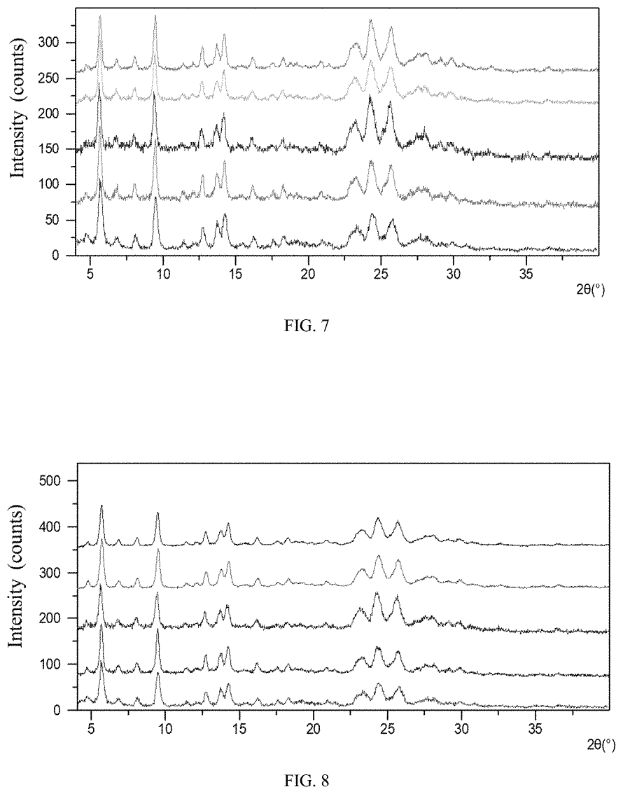

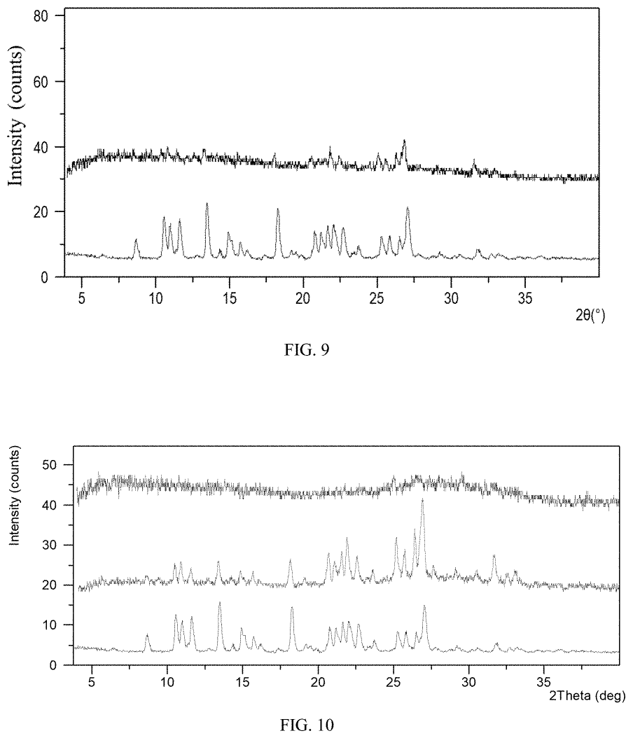

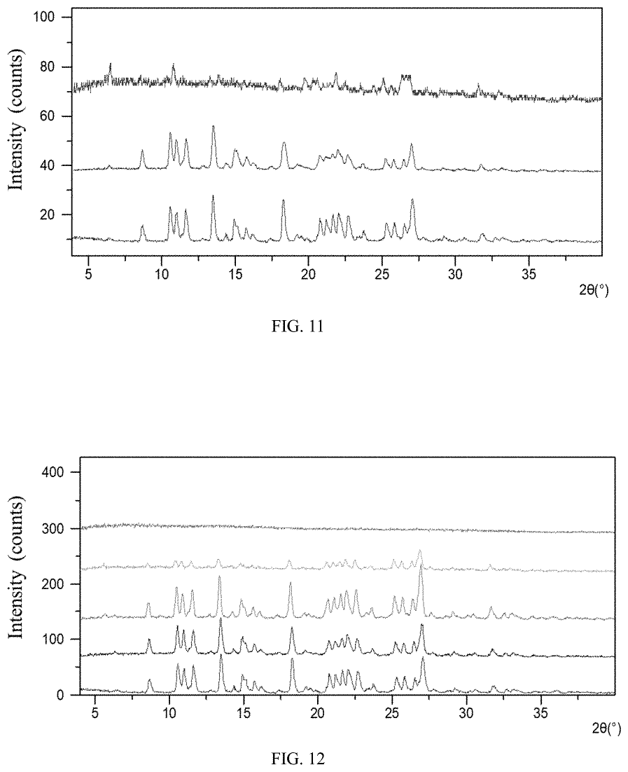

DSC curve is substantially as depicted in FIG. 2, which shows a dehydration endotherm when heated around 69° C. TGA curve is substantially as depicted in FIG. 3, which shows a weight loss of 14.2% when heated to 100° C. Without any limitation being implied, Form K1 is a hydrate.

TABLE 1

| 2θ (±0.2°) | d spacing | Intensity % |
|---|---|---|
| 4.78 | 18.48 | 8.80 |
| 5.75 | 15.38 | 100.00 |
| 6.90 | 12.82 | 16.56 |
| 8.12 | 10.88 | 16.64 |
| 9.50 | 9.31 | 94.77 |
| 11.48 | 7.71 | 15.98 |
| 12.75 | 6.95 | 34.72 |
| 13.80 | 6.42 | 42.93 |
| 14.28 | 6.20 | 70.60 |
| 16.28 | 5.44 | 25.72 |
| 17.66 | 5.02 | 18.50 |
| 18.38 | 4.83 | 32.12 |
| 19.95 | 4.45 | 13.11 |
| 20.99 | 4.23 | 17.58 |
| 23.00 | 3.87 | 24.98 |
| 23.50 | 3.79 | 32.18 |
| 24.58 | 3.62 | 51.94 |
| 25.84 | 3.45 | 49.46 |
| 28.24 | 3.16 | 18.63 |
| 29.22 | 3.06 | 11.93 |
| 30.01 | 2.98 | 15.30 |
| 32.81 | 2.73 | 3.85 |
| 35.57 | 2.52 | 2.70 |
| 36.71 | 2.45 | 3.04 |

### Example 2 Preparation of Form K1

1.0 g of acalabrutinib freebase was weighed into a glass vial, and 10 mL of 1 mol/L HCl aqueous solution was added. After the solid was dissolved, clear solution was obtained through filtration. The obtained clear solution was stirred at 5° C., and 7.9 mL of 1 mol/L NaOH aqueous solution was added at the same time. After the addition of a little crystal seeds, 2.1 mL of NaOH aqueous solution was added. After the mixture was stirred for 2 days, solid was isolated and dried with forced air convention at 35° C. for about 40 hours. The solid obtained in the present example was confirmed to be Form K1. The XRPD data are listed in Table 2.

¹H NMR spectrum of Form K1 is substantially as depicted in FIG. 4, which is consistent with the structure of compound (C₂₆H₂₃N₇O₂). ¹H NMR data are: ¹H NMR (400 MHz, DMSO-d6) δ 10.83 (s, 1H), 8.41 (d, J=4.8 Hz, 1H), 8.22 (d, J=8.3 Hz, 1H), 8.16 (dd, J=8.4, 2.6 Hz, 2H), 7.80 (ddd, J=28.2, 16.5, 8.2 Hz, 4H), 7.23-7.06 (m, 2H), 6.15 (d, J=24.9 Hz, 2H), 5.60 (ddd, J=94.6, 7.6, 4.2 Hz, 1H), 3.82 (t, J=6.6 Hz, 1H), 3.71-3.48 (m, 1H), 2.32 (d, J=9.9 Hz, 2H), 2.18-1.87 (m, 4H), 1.62 (s, 1H).

TABLE 2

| 2θ (±0.2°) | d spacing | Intensity % |
|---|---|---|
| 5.84 | 15.13 | 96.93 |
| 6.94 | 12.74 | 17.67 |
| 8.19 | 10.80 | 18.97 |
| 9.59 | 9.22 | 100.00 |
| 11.56 | 7.66 | 11.69 |
| 12.19 | 7.26 | 7.50 |
| 12.85 | 6.89 | 32.87 |
| 13.85 | 6.40 | 39.31 |
| 14.37 | 6.16 | 70.53 |
| 15.51 | 5.71 | 4.84 |
| 16.37 | 5.41 | 24.75 |
| 17.74 | 5.00 | 17.31 |
| 18.45 | 4.81 | 24.80 |
| 18.95 | 4.68 | 13.77 |
| 21.05 | 4.22 | 14.50 |
| 21.63 | 4.11 | 9.92 |
| 23.46 | 3.79 | 31.53 |
| 24.53 | 3.63 | 56.95 |
| 25.88 | 3.44 | 50.10 |
| 27.80 | 3.21 | 18.40 |
| 28.27 | 3.16 | 17.16 |
| 29.32 | 3.05 | 9.84 |
| 30.09 | 2.97 | 12.54 |

### Example 3 Physical Stability of Form K1 in Water

The following experiments were conducted to compare physical stability of Form K1 of the present disclosure and Form I of WO2017002095A1 in water. Approximately 10 mg of Form K1 and Form I solids were added into 1.0 mL of pure water separately to form suspensions. The suspensions were dispersed by magnetic stirring and shaking at room temperature and 5° C. to evaluate the physical stability of Form K1 and Form I of WO2017002095A1 in water. The results show that no crystalline form change occurs for Form K1 of the present disclosure during magnetic stirring and shaking at room temperature and 5° C., while Form I shows crystallinity decrease and almost converts into amorphous. The results show that Form K1 of the present disclosure has better physical stability in water.

TABLE 3

| Ini- | | | Solid form changes in | | | | |
|---|---|---|---|---|---|---|---|
| tial | | Temper- | the suspension | | | | Fig- |
| form | Method | ature | 4 hours | 1 day | 2 days | 7 days | ures |
| Form | Magnetic | Room | No form | No form | No form | No form | 5 |
| K1 | stirring | temper- | change | change | change | change | |
| | | ature | | | | | |
| | | 5° C. | No form | No form | No form | No form | 6 |
| | | | change | change | change | change | |
| | Shaking | Room | No form | No form | No form | No form | 7 |
| | | temper- | change | change | change | change | |
| | | ature | | | | | |
| | | 5° C. | No form | No form | No form | No form | 8 |
| | | | change | change | change | change | |

TABLE 4

| | | | Solid form changes | | | | |
|---|---|---|---|---|---|---|---|
| Initial | | | in suspension | | | | |
| form | Method | Temperature | 4 hours | 1 day | 2 days | 7 days | Figures |
| Form I | Magnetic | Room | Obvious | N/A | N/A | N/A | 9 |
| | stirring | temperature | crystallinity | | | | |
| | | | decrease | | | | |
| | 5° C. | | Slight | Obvious | N/A | N/A | 10 |
| | | | crystallinity | crystallinity | | | |
| | | | decrease | decrease | | | |
| | Shaking | Room | No form | Obvious | N/A | N/A | 11 |
| | | temperature | change | crystallinity | | | |
| | | | | decrease | | | |
| | | 5° C. | No form | No form | No form | Obvious | 12 |
| | | | change | change | change | crystallinity | |
| | | | | | | decrease | |

### Example 4 Slurry Competition Experiments Between Form K1 and Form I of WO2017002095A1

Approximately 30 mg of Form K1 and Form I of WO2017002095A1 were weighed and suspended into water at 5° C. and room temperature, to evaluate the stability of Form K1 and Form I of WO2017002095A1. Results are listed in Table 5. The results show that Form I converts into Form K1 at both 5° C. and room temperature, suggesting that Form K1 of the present disclosure thermodynamically more stable.

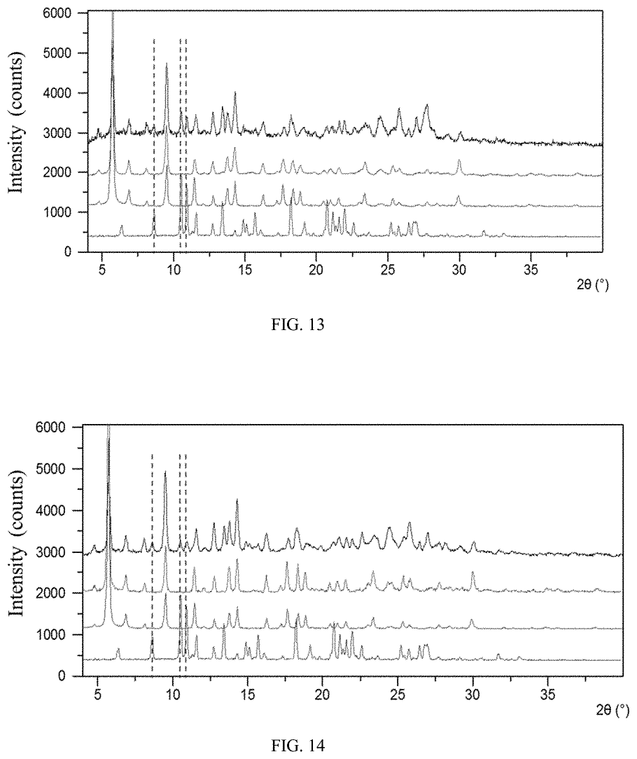

TABLE 5

| | Stirring | Stirring | Solid | Attached |
|---|---|---|---|---|
| Initial form | temperature | time | form | figure |
| Form K1 + Form I | 5° C. | 6 days | Form K1 | FIG. 13 |
| Form K1 + Form I | RT | 24 days | Form K1 | FIG. 14 |

### Example 5 Stability of Form K1 at Long-Term and Accelerated Conditions

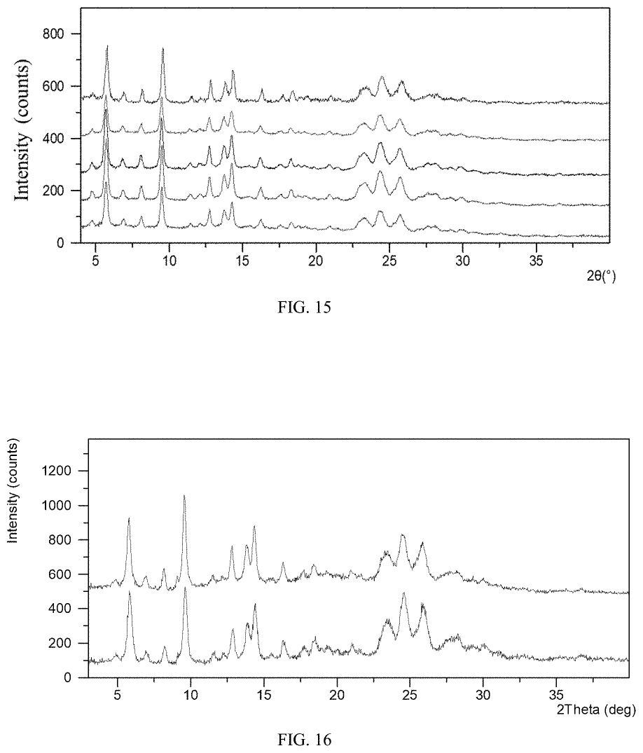

Four samples, 10 mg each of Form K1 of the present disclosure was stored in HPLC glass vials. The HPLC glass vials were capped in sealed conditions, or covered with Parafilm with 3-5 pinholes in open conditions. The samples were stored at conditions listed in table below. After one month, solid forms were checked by XRPD. Test results are shown in Table 6, and the XRPD pattern overlay is shown in FIG. 15.

TABLE 6

| Initial form | Condition | Time | Solid form |
|---|---|---|---|
| Form K1 | 25° C./60% RH, sealed | one month | Form K1 |
| | 40° C./75% RH, sealed | one month | Form K1 |
| | 25° C./60% RH, open | one month | Form K1 |
| | 40° C./75% RH, open | one month | Form K1 |

The results show that Form K1 kept stable for at least one month at 25° C./60% RH (sealed), 25° C./60% RH (open), 40° C./75% RH (sealed) and 40° C./75% RH (open). Therefore, Form K1 has good stability under both long-term and accelerated conditions.

### Example 6 Solubility of Form K1 in Different Buffers

A sufficient amount of Form K1 or Form I of WO2017002095A1 was suspended in buffers of pH 7.0, 7.4 and 8.7. After being stirred and equilibrated for 2 hours, the suspension was centrifuged to obtain supernatant. Concentration (mg/mL) of the supernatant was analyzed by HPLC. Test results are listed in Table 7.

TABLE 7

| | Solubility after equilibration for 2 hours (mg/mL) | |
|---|---|---|
| Buffers | Form K1 | Form I |
| pH = 7.0 phosphate | 0.079 | 0.064 |
| aqueous solution | | |
| pH = 7.4 phosphate | 0.069 | 0.055 |
| aqueous solution | | |
| pH = 8.7 phosphate | 0.066 | 0.056 |
| aqueous solution | | |

The results show that Form K1 has a higher solubility in buffers of pH 7.0, 7.4 and 8.7, compared with Form I of WO2017002095A1.

The absorption of the oral drugs is mainly in the small intestine. It is known that physiological environment in small intestine is almost neutral. Form K1 of the present disclosure has a higher solubility than Form I in neutral or weakly basic medium, which is benefit for successful drug delivery to intestine, as well as drug dissolution and absorption in intestine.

### Example 7: Solubility of Form K1 in Organic Solvents

A certain amount of Form K1 or Form I of WO2017002095A1 was added into a glass vial. Organic solvent was added into the glass vial in increments of 100 μL. Solid was quickly dispersed in solvent through oscillation or sonication, and the process was monitored to see whether the solid was completely dissolved. No more solvent was added when solids were completely dissolved, or a total volume of solvent reached 1.5 mL. According to mass of sample (m) and volume of solvent added, solubility (S) of different crystalline forms in organic solvent was calculated. The results are listed in Table 8.

TABLE 8

| | Solubility | |
|---|---|---|
| | Form K1 | Form I |
| Solvent | (mg/mL) | (mg/mL) |
| Methanol | S > 89.0 | S < 27.0 |
| Ethanol | S > 120.0 | S < 6.4 |
| Isopropanol | S > 48.0 | S < 6.5 |
| n-Propanol | S > 58.0 | S < 7.5 |
| n-Butanol | S > 30.3 | S < 8.7 |
| Acetone | S > 137.0 | S < 6.5 |
| Methyl isobutyl ketone | S > 10.0 | S < 7.7 |
| Methyl ethyl ketone | S > 114.0 | S < 7.6 |
| Ethyl acetate | S > 13.2 | S < 7.9 |
| Tetrahydrofuran | S > 142.0 | S < 16.1 |
| 2-methyltetrahydrofuran | S > 17.6 | S < 8.9 |
| 1,4-Dioxane | S > 102.0 | S < 13.1 |
| Acetonitrile | S > 36.3 | S < 7.8 |

Notes:
the solubility is calculated by S > m/V1 and S < m/V2. V2 is the largest volume of solvent when solid is not completely dissolved, and V1 is the smallest volume of solvent when solid is completely dissolved.

The results show that Form K1 has a higher solubility than Form I in solvents commonly used for crystallization, including methanol, ethanol, isopropanol, n-propanol, n-butanol, acetone, methyl isobutyl ketone, methyl ethyl ketone, ethyl acetate, tetrahydrofuran, 2-methyltetrahydrofuran, 1,4-dioxane and acetonitrile.

### Example 8: Physical Stability of Form K1

Form K1 was ground manually in a mortar for 5 minutes. The XRPD patterns of the solids before and after grinding are shown in FIG. 16. The results show that Form K1 has good physical stability under grinding, as no obvious form change or crystallinity decrease were observed after grinding.

### Example 9: Compressibility of Form K1

Approximately 80 mg of Form K1 or Form I of WO2017002095A1 was added into the dies of φ6 mm round tooling, compressed at 10 kN manually, and then stored in a desiccator for 24 hours until complete elastic recovery. Radial crushing force (hardness, H) was tested with an intelligent tablet hardness tester. Diameter (D) and thickness (L) were tested with a caliper. Tensile strength of the powder with different hardness was calculated with the following formula: T=2H/πDL. Under a certain force, the greater the tensile strength, the better the compressibility. The results are listed in Table 9.

TABLE 9

| Solid form | Tensile strength (MPa) |
|---|---|
| Form I of WO2017002095A1 | Unable to be compressed into a tablet |
| Form K1 | 1.31 |

The results indicate that Form K1 has a higher tensile strength and better compressibility compared with Form I of WO2017002095A1.

### Example 10 Adhesiveness of Form K1

Approximately 30 mg of Form K1 or Form I of WO2017002095A1 was weighed and then added into the dies of φ8 mm round tooling, compressed at 10 kN and held for 30 s. The amount of material sticking to the punch was weighed. The compression was repeated twice and the cumulative amount and average amount of solids sticking to the punch during the compression were recorded. Detailed experimental results are shown in Table 10.

TABLE 10

| Crystal Form | Cumulative amount (μg) | Average amount (μg) |
|---|---|---|
| Form I of | 90 | 45 |
| WO2017002095A1 | | |
| Form K1 | 60 | 30 |

Test results indicate that the amount sticking to the punch of Form I of WO2017002095A1 is higher than that of Form K1. The adhesiveness of Form K1 is superior to that of Form I of WO2017002095A

### Example 11: Formulation Process of Form K1 Drug Product

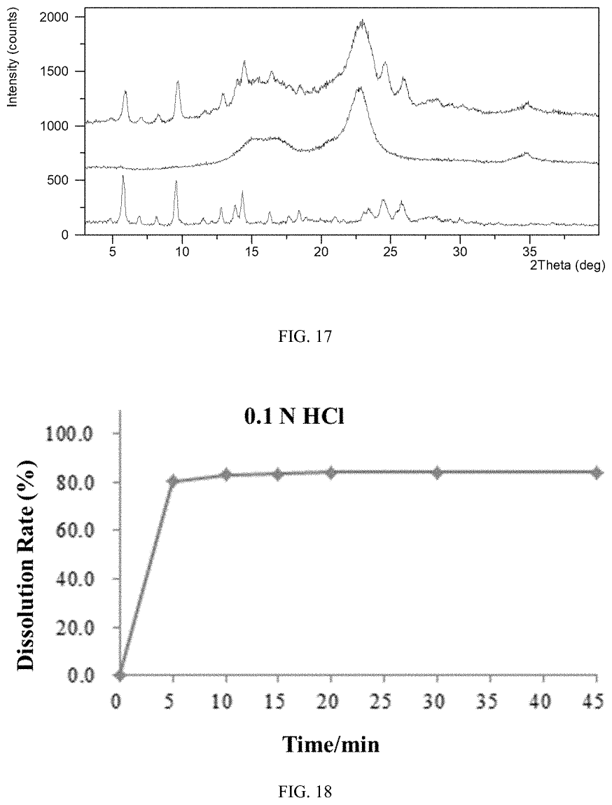

Form K1 capsules were prepared according to the formulation listed in Table 11 and the preparation process described in Table 12. XRPD was performed to test the form change before and after capsule preparation. XRPD results are shown in FIG. 17.

TABLE 11

| No. | Ingredient | Function | mg/capsule | % (w/w) |
|---|---|---|---|---|
| 1 | Form K1 | API | 100.00 | 31.60 |
| 2 | Microcrystalline Cellulose | Filler | 146.83 | 46.40 |
| | (Avicel PH 102) | | | |
| 3 | Partially pregelatinized | Binder | 47.47 | 15.00 |
| | starch | | | |
| 4 | Carboxymethyl starch | Disintegrant | 18.99 | 6.00 |
| | sodium (DST) | | | |
| 5 | Magnesium stearate | Lubricant | 3.16 | 1.00 |
| | (5712) | | | |

| | | |
|---|---|---|
| Total | 316.45 | 100.00 |

TABLE 12

| Step | Procedure |
|---|---|
| Blending | Weigh No. 1-5 materials into an LDPE |
| | bag and blend for 2 minutes |
| Encapsulation | Weigh and fill 316.45 ± 2 mg of |
| | blended powders into the 0# capsules |
| Packaging | Seal one capsule and 1 g of desiccant |
| | into a 35-mL HDPE vial |

The results show that Form K1 doesn't change during the capsule formulation process.

### Example 12: Dissolution Profile of Form K1 in Drug Product

Dissolution test in 0.1 mol/L HCl aqueous solution was performed on Form K1 drug product obtained from example 11. Dissolution method is listed in Table 13. Test results are presented in Table 14 and FIG. 18.

TABLE 13

| Instrument | Agilent 708DS | |
|---|---|---|
| Method | Paddle | |
| Medium | 0.1 mol/L HCl aqueous solution | |
| Volume | 900 | mL |
| Speed | 50 | rpm |

| | |
|---|---|
| Temperature | 37° C. |
| Sampling time | 5, 10, 15, 20, 30 and 45 min |

TABLE 14

| Time (min) | Cumulative dissolution (%) |
|---|---|
| 0 | 0.0 |
| 5 | 80.3 |
| 10 | 83.2 |
| 15 | 83.5 |
| 20 | 84.1 |
| 30 | 84.0 |
| 45 | 84.1 |

As the results show, Form K1 product has a relatively high in vitro dissolution and dissolution rate in 0.1 mol/L HCl aqueous solution. A dissolution of 84% was achieved within 20 minutes.

### Example 13: Stability of Form K1 in Drug Product

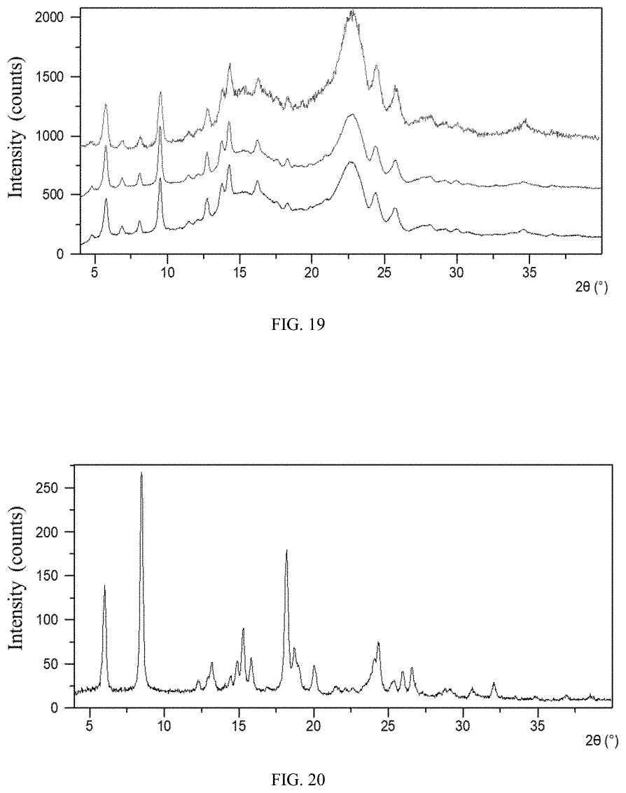

The stability of Form K1 drug product was evaluated with the formulation prepared in Example 11. The capsules were stored under 25° C./60% RH and 40° C./75% RH. After a certain period, XRPD test was performed. Results are presented in Table 15, and the XRPD pattern overlay is shown in FIG. 19.

TABLE 15

| Package | Condition | Time | Solid form |
|---|---|---|---|
| 35 mL HDPE vial + 1 | 25° C./60% RH | 6 months | No form change |
| g of desiccant | 40° C./75% RH | 6 months | No form change |

The results show that Form K1 drug product is stable for at least 6 months at 25° C./60% RH and 40° C./75% RH.

### Example 14 Preparation of Form CS10

20 g of acalabrutinib freebase was weighed and dissolved in 200 mL of acetonitrile/MeOH (9:1, v/v). After the mixture was stirred and heated to 50° C., solid was precipitated and isolated. 50 mg of the obtained solid was weighed into a 1.5-mL glass vial and 1 mL of isopropyl acetate was added. The mixture was stirred at 5° C. for 20 hours and white crystalline solid was obtained.

The obtained white crystalline solid obtained in the present example is confirmed to be Form CS10. The XRPD pattern is substantially depicted in FIG. 20, and the XRPD data are listed in Table 16.

TABLE 16

| 2θ (±0.2°) | d spacing | Intensity % |
|---|---|---|
| 6.03 | 14.67 | 48.76 |
| 8.52 | 10.38 | 100.00 |
| 12.28 | 7.21 | 5.06 |
| 13.19 | 6.71 | 14.04 |
| 14.91 | 5.94 | 14.72 |
| 15.31 | 5.79 | 29.95 |
| 15.82 | 5.60 | 16.47 |
| 18.21 | 4.87 | 67.09 |
| 18.71 | 4.74 | 21.77 |
| 20.03 | 4.43 | 13.55 |
| 21.50 | 4.13 | 3.67 |
| 24.40 | 3.65 | 23.73 |
| 25.35 | 3.51 | 7.00 |
| 25.97 | 3.43 | 11.64 |
| 26.55 | 3.36 | 13.32 |
| 29.05 | 3.07 | 3.09 |
| 30.61 | 2.92 | 3.86 |
| 32.07 | 2.79 | 6.80 |

### Example 15 Preparation of Form CS10

20 g of acalabrutinib freebase was weighed and then dissolved in 200 mL of acetonitrile/MeOH (9:1, v/v). After the mixture was stirred and heated to 50° C., solid was precipitated and isolated. 310 mg of the obtained solid was weighed into a 50-mL crystallizer and 15 mL of isopropyl acetate/dichloromethane (95:5, v/v) was added. After addition of about 2 mg of Form CS10 as seed, the mixture was slurred at 5° C. for 21 hours and white crystalline solid was obtained.

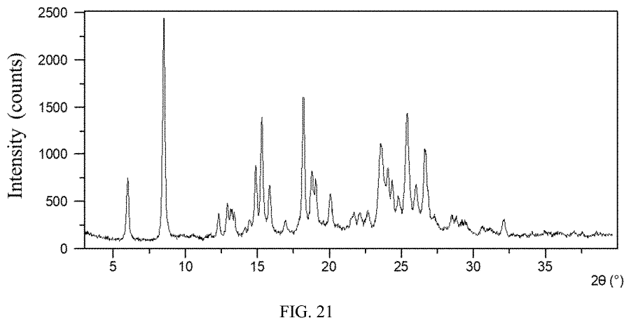

The obtained white crystalline solid obtained in the present example is confirmed to be Form CS10. The XRPD pattern is substantially as depicted in FIG. 21, and the XRPD data are listed in Table 17.

TABLE 17

| 2θ (±0.2°) | d spacing | Intensity % |
|---|---|---|
| 6.08 | 14.54 | 57.84 |
| 8.52 | 10.38 | 100.00 |
| 12.34 | 7.17 | 4.78 |
| 13.25 | 6.68 | 14.17 |
| 14.50 | 6.11 | 8.22 |
| 14.89 | 5.95 | 12.87 |
| 15.36 | 5.77 | 23.48 |
| 15.88 | 5.58 | 19.54 |
| 18.26 | 4.86 | 97.57 |
| 18.71 | 4.74 | 22.32 |
| 19.09 | 4.65 | 12.23 |
| 20.05 | 4.43 | 17.53 |
| 21.53 | 4.13 | 4.79 |
| 22.72 | 3.91 | 3.37 |
| 23.36 | 3.81 | 5.96 |
| 24.08 | 3.70 | 25.00 |
| 24.41 | 3.65 | 39.61 |
| 25.42 | 3.50 | 12.55 |
| 26.02 | 3.42 | 23.08 |
| 26.59 | 3.35 | 22.77 |
| 28.79 | 3.10 | 5.34 |
| 29.20 | 3.06 | 6.70 |
| 30.64 | 2.92 | 7.83 |
| 32.08 | 2.79 | 12.46 |
| 34.92 | 2.57 | 2.23 |
| 36.98 | 2.43 | 3.97 |
| 38.58 | 2.33 | 3.11 |

The examples described above are only for illustrating the technical concepts and features of the present disclosure, and intended to make those skilled in the art being able to understand the present disclosure and thereby implement it, and should not be concluded to limit the protective scope of this disclosure. Any equivalent variations or modifications according to the spirit of the present disclosure should be covered by the protective scope of the present disclosure.

1. A crystalline form K1 of acalabrutinib, 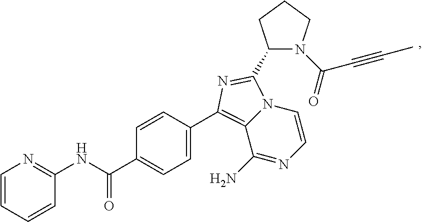 [SMILES: CC#CC(=O)N1CCC[C@H]1c1nc(-c2ccc(C(=O)Nc3ccccn3)cc2)c2c(N)nccn12]
wherein the X-ray powder diffraction pattern shows characteristic peaks at 2theta values of 5.8°±0.2°, 9.5°±0.2°, and 14.3°±0.2° using CuKα radiation.

2. The crystalline form K1 of acalabrutinib according to claim 1, wherein the X-ray powder diffraction pattern shows one or two or three characteristic peaks at 2theta values of 13.8°±0.2°, 12.8°±0.2°, and 18.4°±0.2° using CuKα radiation.

3. The crystalline form K1 of acalabrutinib according to claim 1, wherein the X-ray powder diffraction pattern shows one or two or three characteristic peaks at 2theta values of 16.3°±0.2°, 6.9°±0.2° and 11.5°±0.2° using CuKα radiation.

4. A process for preparing crystalline form K1 of acalabrutinib according to claim 1, wherein the process comprises:
(1) Adding acalabrutinib freebase and an acid in a mixture of a ketone and water, stirring, separating and drying to obtain a solid, suspending the above solid in water and adding an alkaline solution into the suspension while stirring, separating the solid to obtain Form K1; or
(2) Adding acalabrutinib free base in an acid solution, stirring, separating and drying to obtain a solid, suspending the solid in an alkaline solution, separating the solid to obtain Form K1.

5. The process according to claim 4, wherein in method (1), said acid is maleic acid or fumaric acid, said alkaline solution is a NaOH aqueous solution, said ketone solvent is acetone or 2-butanone or methyl isobutyl ketone; In method (2), said acid solution is an aqueous solution of HCl, said alkaline solution is an aqueous solution of NaOH.

6. A pharmaceutical composition, said pharmaceutical composition comprises a therapeutically effective amount of crystalline form K1 of acalabrutinib according to claim 1, and a pharmaceutically acceptable carrier, a diluent or an excipient.

7. (canceled)

8. A method of treating mantle cell lymphoma, comprising administering to a subject in need thereof a therapeutically effective amount of crystalline form K1 of acalabrutinib according to claim 1.

9. The crystalline form K1 of acalabrutinib according to claim 2, wherein the X-ray powder diffraction pattern shows one or two or three characteristic peaks at 2theta values of 16.3°±0.2°, 6.9°±0.2° and 11.5°±0.2° using CuKα radiation.

10. A pharmaceutical composition, said pharmaceutical composition comprises a therapeutically effective amount of crystalline form K1 of acalabrutinib according to claim 2, and a pharmaceutically acceptable carrier, a diluent or an excipient.

11. A method of treating chronic lymphocytic leukemia, comprising administering to a subject in need thereof a therapeutically effective amount of crystalline form K1 of acalabrutinib according to claim 1.

12. A method of treating macroglobulinemia, comprising administering to a subject in need thereof a therapeutically effective amount of crystalline form K1 of acalabrutinib according to claim 1.

13. A method of treating follicular lymphoma, comprising administering to a subject in need thereof a therapeutically effective amount of crystalline form K1 of acalabrutinib according to claim 1.

14. A method of treating diffuse large B cell lymphoma, comprising administering to a subject in need thereof a therapeutically effective amount of crystalline form K1 of acalabrutinib according to claim 1.

15. A method of treating multiple myeloma, comprising administering to a subject in need thereof a therapeutically effective amount of crystalline form K1 of acalabrutinib according to claim 1.

16. A pharmaceutical composition, said pharmaceutical composition comprises a therapeutically effective amount of crystalline form K1 of acalabrutinib according to claim 9, and a pharmaceutically acceptable carrier, a diluent or an excipient.
